(19) United States
(12) Patent Application Publication
(10) Pub. No.: US 2018/0221131 A1
(43) Pub. Date: Aug. 9, 2018

(54) DIVERTER FOR REDUCING TURBULENT FLOW IN AN ANEURYSM, FISTULA AND THE LIKE

(71) Applicant: Steven J. Ferry, Excelsior, MN (US)

(72) Inventor: Steven J. Ferry, Excelsior, MN (US)

(21) Appl. No.: 15/429,895

(22) Filed: Feb. 10, 2017

Related U.S. Application Data
(63) Continuation of application No. 62/285,033, filed on Sep. 26, 2016.

(51) Int. Cl. A61F 2/06; A61F 2/90; A61L 31/02; A61L 31/14; A61L 31/04; A61L 31/18; D04B 1/22; D04B 21/20

(52) U.S. Cl. CPC A61F 2/06; A61F 2/90; A61L 31/022; A61L 31/14; A61F 2002/823; A61L 31/18; D04B 1/225; D04B 21/205; A61F 2002/068; A61L 31/04

(57) ABSTRACT

Embodiments disclosed herein include a knitted diverter that includes a main body defined by wire defining struts and loops having a density that reduces flow through an aneurysm, fistula or other similar anomaly.

### PRIORITY

This Application claims the benefit of U.S. Provisional Application Ser. No. 62/285,033, filed Sep. 26, 2016, and U.S. application Ser. No. 15/275,718, filed Sep. 26, 2016, which applications are incorporated by reference herein their entireties.

### FIELD

Inventive subject matter disclosed herein relates to a diverter for reducing turbulent flow in an aneurysm, fistula and similar physiological anomalies.

### BACKGROUND

Conventional methods of slowing or diverting flow of blood into aneurysms include placing the diverter within a parent artery in such a way so that the diverter crosses a neck of an aneurysm which has formed off of the parent artery. These conventional diverter devices are braided and are sometimes formed from multiple materials such as Pt, Co, NiTi, as well as others. One difficulty experienced with these types of flow diverters has been the incidence of latent complications such as aneurysm rupture or emboli which form on a device surface and releases downstream resulting in an ischemic blockage.

### SUMMARY

Embodiments disclosed herein include a knitted diverter that includes a main body defined by wire defining struts and loops having a density that reduces flow through an aneurysm, fistula or other similar anomaly.

### DESCRIPTION OF DRAWINGS

FIG. 1 illustrates a top plan view of one embodiment of a diverter of the present invention.

FIG. 2 illustrates a perspective view of a diverter embodiment, illustrating struts and loops.

FIG. 3 illustrates a perspective view of a coated diverter embodiment.

FIG. 4 illustrates a top plan view of another diverter embodiment.

FIG. 5 illustrates a top plan view of another diverter embodiment.

### DETAILED DESCRIPTION

The following detailed description includes references specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Inventive embodiments disclosed herein include a knitted occlude, also referred to as a knitted diverter, for diversion of flow within vessels of the heart, brain, hepatics, lumbar, pancreatic and the like. Inventive embodiments disclosed herein include a knitted luminal device or a knitted shaped device which when placed in an artery reduce flow force into a malformation such as an aneurysm. The invention may be fabricated as a luminal knitted device or flat knit and then shaped using methods known in the art.

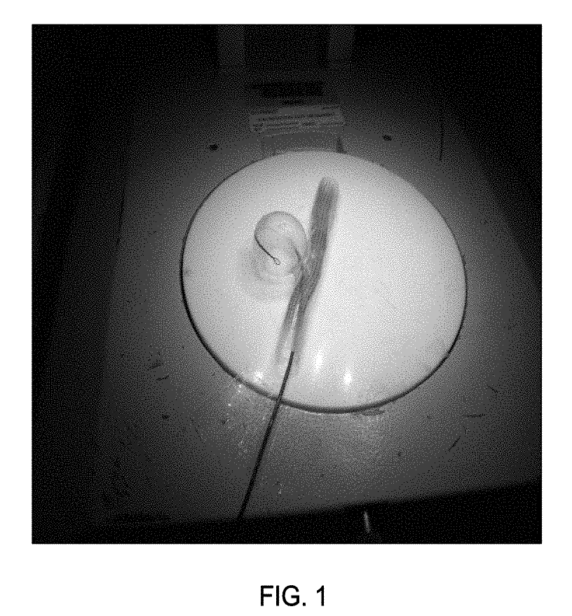

Inventive embodiments disclosed for one embodiment at 10 in FIG. 1 include a diverter 10 having a main body 12 having a knitted luminal configuration 14 of varying Inner Diameter/Outer Diameter ratios. Materials used to knit the diverter main body include metals and polymers. The metals are of varying diameters and are in either a solid wire or polymer of tubular configuration. Polymers include phase transition polymers. As used herein, the term “phase transition polymer” refers to a polymer that changes in response to external stimuli, such as pH, temperature, light, metabolite and electrical current. The length of the knitted diverter embodiments is varied depending on the diameter of a given vessel into which the diverter is subsequently inserted. Loop configuration embodiments are alterable as well, resulting in a tighter or more open knitted mesh.

Configuration embodiments of the mesh, one of which is shown at 20 in FIG. 1A are alterable to provide varied geometries. Mesh patterns of embodiments disclosed herein determine the amount of flow reduction into and out of an aneurysm.

Inventive embodiments disclosed herein include a flexible luminal or shaped knitted diverter for placement in locations within the human vasculature for the treatment of various disorders by Interventionalists. The luminal diverter has an inner diameter ranging from 0.004-0.10 mm to 0.495-12.4 mm and an outer diameter ranging from 0.008-0.2 mm to 0.50-12.5 mm. The shaped knitted diverter has a diameter 359°≤. When inserted in blood vasculature, diverter embodiments allow blood to flow through with reduced pressure and turbulence an aneurysm, reducing the potential for the aneurysm to rupture.

In one embodiment, the diverter is knitted to have a luminal configuration. The ends of the diverter are finished with conventional loops or a “Lock Stitch”. Materials for fabrication of the knitted diverter embodiment include metal wire, both flat and round, metal tubing, polymeric filaments as well as polymeric tubing.

In another embodiment, the knitted diverter is flat knit and then shaped into a semi-circular configuration on a mandrel or other such techniques known in the art. The radial force that the diverter applies to the wall of a vessel is determined by the selected knitting material as well as the radius of the mandrel on which it is shaped. If, for example, a shaped knitted diverter is to be placed into a four mm parent artery, then the diverter is shaped on a five mm mandrel, allowing for expansion force to be exerted on the vessel wall by the diverter. Determining the optimal ratio of diverter outer diameter to-blood vessel inner diameter is determined by examining the patency of the vessel wall relative to the amount of radial force exerted by the shaped knitted diverter, which is measured in both cases.

Inventive embodiments employ a round or flat wire or both round and flat wires which are composed of nitinol, titanium, stainless steel or other suitable metals that are capable of being placed on a knitting machine. Conversely, the fiber embodiments are fabricated from a suitable polymer.

Marker bands are included on either end of a diverter. These marker bands are used by the physician to gauge distance between the proximal and distal ends of the diverter. For other embodiments, a palladium wire or other radiopaque material is co-knitted to form the diverter. A radiopaque wire is integrated into the fabrication of the diverter by becoming one of the elements knitted. For other embodiments, multiple radiopaque wires are utilized to enhance radiopacity. The purpose of the diverter is to reduce turbulent flow into a vessel abnormality to reduce a risk of aneurysm of fistula rupture.

Some luminal diverter embodiments are self expanding and are reducible in diameter by methods of knitting known to those knowledgeable in the art. These knitted diverter embodiments are inserted into a luminal shaft such as a catheter for controlled delivery to a target site. These knitted diverter embodiments include radiopaque markers on each end. For other embodiments, a very low or non-thrombogenic radiopaque wire is a portion of the knit configuration. Some knitted diverter embodiments are also be plated with a radiopaque material in part or in whole. A shaped diverter is fabricated into a variety of shapes such as round, elliptical, elongated and special shapes which are formed by heat setting the mesh material on a mandrel.

Mesh employed in diverter embodiments disclosed herein normally knit from wires ranging in diameter from 0.0015″ to 0.0200″. For some embodiments, metal textiles have knit wires ranging in diameter from 0.0005″ to 0.0350″. These wires are round or flat such as those used in metal textiles copper gauze for cleaning and in air-filtration applications where large surface areas of wire are needed. A round wire when flattened has approximately twice as much surface area.

In flow diversion applications, wire diameter is perhaps the most important design variable. It directly affects flow, pressure drop and cost. Therefore, a balance must be struck between using a higher density diverter of higher cost fine wire or a lesser density diverter using lower cost heavy wire.

Loop density refers to mass of material per unit volume. It is related to the density of the original mesh and the amount of forming pressure used to compress the mesh into its final form. Determining the required mesh density to produce the specified final product density is a critical step in the design process. Proper density ensures optimal performance of the flow reduction application.

The main body 12 of the knitted flow diverter includes, for some embodiments, a metal tube or wire or appropriate polymeric material, all of which are of a low thrombogenic nature.

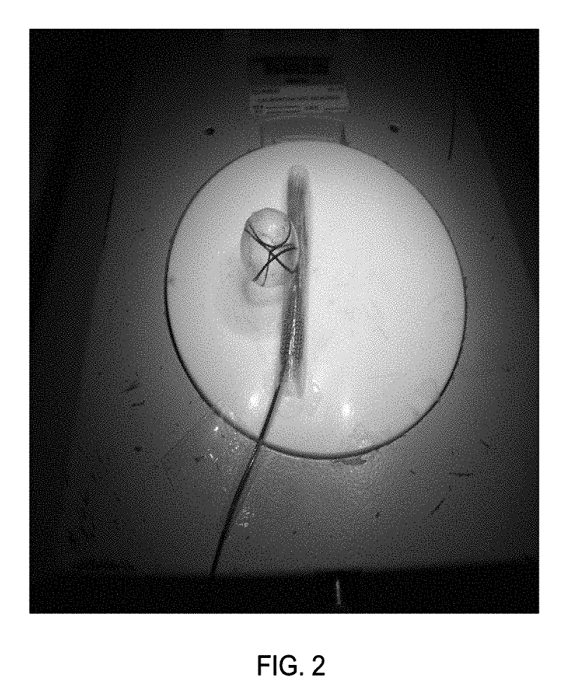

The geometry of the knitted diverter embodiments disclosed herein enables flow disruption more readily than does geometry of conventional braided diverters. One key to flow disruption, slowing the flow into an aneurysm, reducing internal turbulence and pressure, is the configuration of the mesh itself. The present inventive embodiments utilize mesh configurations and densities that employ the positioning of the strut 22, shown in FIG. 1A as perpendicular, when deployed within a parent artery, relative to the aneurysm neck and natural flow of blood within the parent artery. When the blood contacts the perpendicular strut 22, the blood is slowed and flows into the aneurysm with much less force that if the aneurysm neck were not covered. Similarly, the linearity of a braided diverter does not inhibit flow as effectively due to the orientation of the braid pics or holes, which are parallel with both the arterial flow and the orientation of the aneurysm neck. Another embodiment showing a strut 22 and loop 23 structure is shown at 20 in FIG. 2.

Another feature of the loop density of is the ability to alter the orientation of the mesh/struts and loop density in a manner that gives control over the amount and direction of flow into the aneurysm.

A radiopaque wire may be integrated into the knitting process for visualization under fluoroscopy or the knitted diverter may have proximal and distal radiopaque markers, or may be completely or selectively plated with an appropriate radiopaque material.

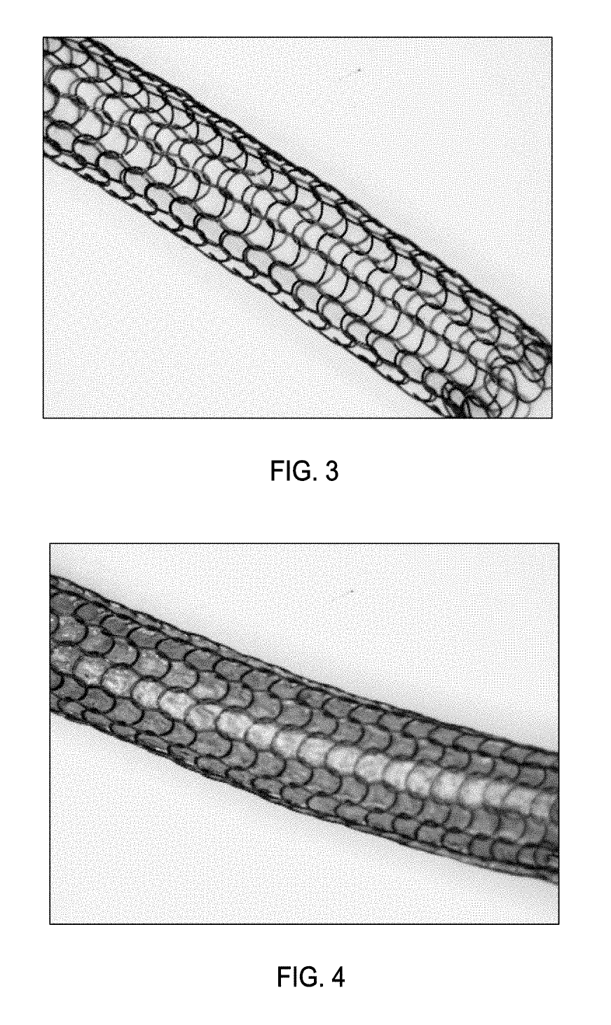

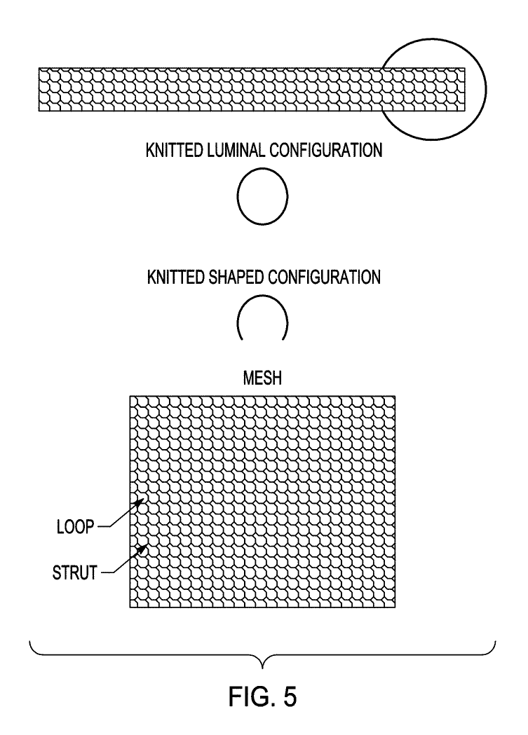

For some embodiments, the diverter is coated, as shown at 30 in FIG. 3. Additional embodiments are shown in FIGS. 4 and 5.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description

1. A knitted diverter comprising:
A main body defined by wire defining struts and loops having a density that reduces flow through an aneurysm, fistula or other similar anomaly.

2. The diverter of claim 1, wherein the wire is metal.

3. The diverter of claim 1, wherein the wire is plastic.

4. The diverter of claim 1, wherein the wire is a phase transition polymer.

5. The knitted diverter of claim 1, wherein the diverter is defined by at least one end having a lock stitch.

6. The knitted diverter of claim 1, wherein the main body includes one or more marker bands.

7. The knitted diverter of claim 1, further comprising a coating that coats the main body.

8. The knitted diverter of claim 1 wherein the struts are perpendicular to blood flow when the diverter is installed adjacent an anomaly.
